(19) United States
(12) Patent Application Publication
(10) Pub. No.: US 2023/0309908 A1
(43) Pub. Date: Oct. 5, 2023

(54) SYSTEM FOR SCREENING TISSUE ON THE PRESENCE OF MALIGNANT CELLS

(71) Applicant: Teledyne Dalsa B.V., Eindhoven (NL)

(72) Inventors: Gerard Johan Den Heeten, Enschede (NL); Ivo Jan Christian Aarninkhof, Enschede (NL)

(21) Appl. No.: 18/295,067

(22) Filed: Apr. 3, 2023

(30) Foreign Application Priority Data
Apr. 4, 2022 (NL) 2031483

(51) Int. Cl. A61B 5/00; G01N 29/11

(52) U.S. Cl. CPC A61B 5/4312; G01N 29/11; G01N 2291/044; G01N 2291/02475

(57) ABSTRACT

A system for screening tissue on the presence of malignant cells in said tissue, which system comprises a wave detector and a data processing device connected or connectable to the wave detector for processing data received from the wave detector, which system comprises an actuator to excite the tissue which is suspected to comprise malignant cells, and the data processing device comprises an analyzer connected to the wave detector for analyzing the data received from the wave detector in response to the actuator exciting the tissue, which analyzer is arranged to identify the tissue with an elevated probability to comprise malignant cells in comparison with tissue that is not suspected to comprise malignant cells in an individual case. The inventors remark that the identification of high but symmetrical outcome results can be used in a population-based stratification of risk of developing malignant cells and can serve as a biomarker in risk-based screening approaches.

### CROSS REFERENCE TO RELATED APPLICATIONS

This application claims priority to and the benefit of Dutch Patent Application No. NL2031483 filed Apr. 4, 2022 and entitled “System for screening tissue on the presence of malignant cells” which is incorporated herein by reference in its entirety.

### TECHNICAL FIELD

The invention relates to a system for screening tissue on the presence of malignant cells in said tissue, which system comprises a wave detector and a data processing device connected or connectable to the wave detector for processing data received from the wave detector.

### BACKGROUND

Although the invention is not restricted thereto, examples of screening tissue on the presence of malignant cells can in particular be found in the field of mammography. Examples of mammography devices and methods for processing mammography images are for instance provided by US2012/033786 (Zinreich, US2015/139523 (EBM Technologies), US2021/315533 (Vieworks Co Ltd), JP2014036906 (Toshiba), KR20150070722 (Samsung), US2010/104063 (Gen. Electric), CN 110364250 (University of Shenshen) and JP2016106963 (Canon), just to name a few of the 15,320 searchable hits on the topic of ‘mammography’ provided on espacenet.com. On ‘detection of malignant cells or tissue’ in general there are even more than 74,000 searchable hits. In other words the skilled person in the field of tissue screening in search of malignant cells is overloaded with information how this can be accomplished.

### SUMMARY

A prominent disadvantage of prior art systems and methods is that they are either invasive, or require educated and trained personnel to carry out those prior art methods, or are unpleasant for the patient, or subject the patient to for instance x-ray imaging which in itself may pose health risks.

It is an object of the invention to propose a system and method in which these disadvantages are at least in part alleviated or mitigated.

According to the invention a system and method is proposed with the features of one or more of the appended claims. In a first aspect of the invention the system comprises an actuator to mechanically excite the tissue which is suspected to comprise malignant cells, and the data processing device comprises an analyzer connected to the wave detector for analyzing the data received from the wave detector in response to the actuator mechanically exciting the tissue, which analyzer is arranged to identify the tissue with an elevated probability to comprise malignant cells in comparison with tissue that is not suspected to comprise malignant cells.

According to a first aspect of the present disclosure there is provide a system for screening tissue on the presence of malignant cells in said tissue, which system comprises a wave detector and a data processing device connected or connectable to the wave detector for processing data received from the wave detector. The system comprises an actuator to mechanically excite the tissue which is suspected to comprise malignant cells, and the data processing device comprises an analyzer connected to the wave detector for analyzing the data received from the wave detector in response to the actuator mechanically exciting the tissue, which analyzer is arranged to identify the tissue with an elevated probability to comprise malignant cells in comparison with tissue that is not suspected to comprise malignant cells.

The analyzer may determine from the data received from the wave detector an estimated stiffness value of the tissue under investigation, wherein the analyzer is arranged to use this stiffness value as an indication for the presence or nonpresence of malignant cells.

The analyzer may determine from the data received from the wave detector local differences of estimated stiffness values of the tissue under investigation, wherein the analyzer is arranged to use these local differences of stiffness values as an indication for the presence or nonpresence of malignant cells.

The data processing device may comprise or connect to a data storage device for storage of data received from the wave detector.

The analyzer may be equipped to operate on data received from the wave detector and/or from the data storage device, wherein the data has different timestamps so as to enable a longitudinal comparison of data over time and to derive from this comparison an indication of the probable presence of malignant cells.

The actuator may be one of a sound emitter, an ultrasound emitter, a pulsed laser source, a displacement actuator.

The actuator may be a displacement actuator for displacing a tissue engaging surface along a predetermined path and/or applying a predetermined load on the tissue to be investigated.

The wave detector may be arranged to collect and provide data to the data processing device, said data relating at least to the tissue being disengaged and released from a load by the displacement actuator, and to the tissue being engaged and loaded by the displacement actuator to a predetermined full load.

The wave detector may be at least one of an (ultra-) sound wave detector, a visual light spectrum 2D or 3D static picture camera, a visual light spectrum 2D or 3D moving picture camera, an infrared detector, an ultraviolet detector, a LIDAR, a radar, a microwave antenna.

The camera may be a CCD-camera.

The displacement actuator may be equipped with at least one marker which enables position detection of the tissue engaging surface or contact area of the displacement actuator.

A further detectable marker may be provided on the supporting surface against which the displacement actuator presses the tissue under investigation.

The wave detector may be arranged to detect the position of the marker.

The data collected and provided by the wave to the data processing device may relate to a complete load-path-trajectory wherein the load provided by the displacement actuator on the tissue develops from no-load to the predetermined full load.

The data processing device may be equipped to estimate from the data a contact area between the displacement actuator and the tissue during engagement of the tissue by the displacement actuator.

The displacement actuator may be equipped with a capacitive measurement organ to determine a contact area between the displacement actuator and the tissue during engagement of the tissue by the displacement actuator.

The analyzer may be arranged to derive an estimated stiffness value of the tissue from the estimated or measured contact area between the displacement actuator and the tissue during engagement of the tissue by the displacement actuator, or from a longitudinal comparison of the estimated or measured contact area developing over time and to derive from this comparison an indication of the presence of malignant cells.

The estimation or measurement of the contact area between the displacement actuator and the tissue may be monitored during a complete cycle of engaging and disengaging of the tissue by the displacement actuator.

The analyzer may be arranged to derive an estimated stiffness value of the tissue from a measured or calculated force applied to the tissue by the displacement actuator and the estimated or measured contact area between the displacement actuator and the tissue during engagement of the tissue by the displacement actuator.

The analyzer may be arranged to derive an estimated stiffness value of the tissue from a longitudinal comparison of a measured or calculated force applied to the tissue by the displacement actuator over time, and the estimated or measured contact area developing over time between the displacement actuator and the tissue during engagement of the tissue by the displacement actuator.

The data processing device may be equipped to determine from the data an estimated volume of the tissue being investigated.

The data processing device may be equipped to determine from the data an estimated volume of the tissue being investigated when the displacement actuator is inactive and/or when the displacement actuator applies a load on the tissue and/or during a complete cycle of engaging and disengaging of the tissue by the displacement actuator.

The analyzer may be arranged to derive an estimated stiffness value of the tissue from a measured or calculated force applied to the tissue and at least one of the estimated volume of the tissue, the contact area between the displacement actuator and the tissue, and a projected area obtained from a projection of the volume of the tissue, one thing and another derived from the data when the displacement actuator is inactive and/or when the displacement actuator applies a constant load to the tissue, and/or from the data when the displacement actuator applies an increasing load on the tissue.

The system may be equipped with a position sensor for measuring a position of a tissue engaging surface or contact area of the displacement actuator and/or a force sensor for measuring a force that the displacement actuator provides to the tissue, which position sensor and/or force sensor are connected to the data processing device for use by the data processing device in combination with the estimated or measured contact area between the displacement actuator and the tissue during engagement of the tissue by the displacement actuator so as to arrange that the load applied by the displacement actuator to the tissue is normalized to a pressure which the displacement actuator applies to the tissue.

The system may be designed for processing data derived from a humans breast.

The system may be designed for processing data derived from a left breast and a right breast from a same patient, and arranging the analyzer to derive from differences between the left breast and the right breast an indication of malignant cells in one breast or both breasts.

The systems and methods disclosed herein may be based on the thought that developing tumors can already be felt or seen, or measured by ultrasound for instance, although in conventional screening it is more common to apply imaging modalities. The system and method of the invention can thus to a certain extent narrow the gap between a physical examination and imaging. A further benefit is that the invention provides the system with the capability (but not the necessity) to automatically identify tissue with malignant cells, which opens the possibility (but again: not the necessity) to apply the method and at least part of the system in the privacy of one's own home, while the system entertains a remote connection between the (local) wave detector and the (remotely positioned) data processing device.

The term ‘wave or waves’ is used to express the generality of the type of detector and in connection therewith the type of actuator that may be used. It is for instance proposed that the actuator is one of a sound emitter, an ultrasound emitter, a pulsed laser source (which inflicts ultrasound waves in the tissue), or simply a displacement actuator. On the other hand the detector is preferably one of an (ultra-)sound wave detector, a visual light spectrum 2D or 3D static picture camera, a visual light spectrum 2D or 3D moving picture camera, an infrared detector, an ultraviolet detector, but it can also be a LIDAR, a radar, or a microwave antenna. When using a camera, which can be regarded as an electromagnetic wave detector, it is preferred that this is a CCD camera, which makes digital processing of data from the camera received by the data processing device easy.

Since the singular embraces the plural, it is evident that there may also be more than one detector, or even detectors of different design and functionality. To stipulate that all types of detector may be used, the following discussion will consistently refer to ‘detector’ or ‘wave detector’, which may then be understood as any one of the indicated types of detector.

An important aspect of the system and method of the invention is that the analyzer determines from the data received from the wave detector an estimated stiffness value of the tissue under investigation, wherein the analyzer is arranged to use this stiffness value as an indication for the presence or nonpresence of malignant cells. This is based on the view of the inventors that the presence of malignant cells in certain tissue also leads to changed mechanical properties of such tissue, and that monitoring these mechanical properties makes possible to accomplish potentially a more early detection of the malignant cells in a significant amount of cases where prior art modalities fail. In such prior art screening modalities this information is not used.

In a certain embodiment of the system and method of the invention it is desirable to fine-tune the detection of the malignant cells by using the data from the wave detector, and to arrange that the analyzer determines local differences of estimated stiffness values of the tissue under investigation, wherein the analyzer is arranged to use these local and/or asymmetrical differences of stiffness values as an indication for the presence or nonpresence of malignant cells. The above-mentioned local differences are considered to differentiate between diseased tissue and healthy tissue.

In some embodiments it is helpful that the data processing device comprises or connects to a data storage device for storage of data from the wave detector. This makes a comparison of current data with data stored in the data storage device possible, which can be helpful in determining differences in the collected data over time, which may also be an indication for the occurrence of malignant cells. It is then preferable that the analyzer is equipped to operate on data from the wave detector and/or from the data storage device, wherein the data has different timestamps so as to enable a longitudinal comparison of data over time and to derive from this comparison an indication of the presence of malignant cells.

A beneficial way of using the system and method of the invention is that the system is designed for processing data derived from a humans breast. In such a design the system and method of the invention may add crucial information to the information derived with a conventional mammography apparatus and method, or be used in addition to a conventional mammography apparatus and method, so as to increase the reliability of detecting malignant cells. The following disclosure is best understood as a system and method to investigate a humans breast, although the system and method can be applied more broadly, also for the detection of malignant cells in other tissue

Preferably the system comprises a displacement actuator for displacing a tissue engaging surface or contact area along a predetermined path and/or applying a predetermined load on the tissue to be investigated. This is an effective way of mechanically exciting the tissue so that the detector can be arranged to collect and provide data to the data processing device, said data relating at least to the tissue being disengaged and released from a load by the displacement actuator, and to the tissue being engaged and loaded by the displacement actuator (in the complete path up) to a predetermined full load. Comparing the data gathered from the tissue in the non-loaded situation with the data from the tissue in the loaded situation, and preferably also in the path between the unloaded and the loaded situation, can then be used to derive an indication of the presence of malignant cells. For clarity it is further mentioned that the displacement actuator can be a static actuator, but also a dynamic actuator in the sense that the displacement actuator produces vibrations in addition to moving from disengagement until engagement of the tissue to be investigated.

To make processing of the data, in particular position data of the tissue engaging surface or contact area of the displacement actuator more easy, it is preferable that the displacement actuator is equipped with at least one marker which enables position detection of the tissue engaging surface or contact area. Detection of the marker is then easy by arranging that the detector is equipped for detection of electromagnetic waves, which may be visual or nonvisual waves. It may be advantageous to provide a further detectable marker on the supporting surface against which the displacement actuator presses the tissue under investigation.

In some embodiments it is useful that the data collected and provided by the wave detector to the data processing device relate to a complete load-path-trajectory wherein the load provided by the displacement actuator on the tissue develops from no-load to the predetermined full load. The accuracy and reliability of the detection of malignant cells can thus be increased.

Surprisingly the inventors have found that it may already suffice for the detection of malignant cells that the data processing device is equipped to estimate a contact area between the displacement actuator and the tissue during engagement of the tissue by the displacement actuator. The extent of this contact area can already be seen as an indirect measure for the tissue stiffness.

Further, stiffness differences as such are found to be strongly indicative for the existence or occurrence of malignant cells in an individual case. When looking at a population, evidence exists that stiffness as such can be used as a biomarker, comparable with mammographic density. The inventors remark that the identification of high but symmetrical outcome results can be used in a population-based stratification of risk of developing malignant cells and can serve as a biomarker in risk-based screening approaches.

More reliable results than with estimation can be achieved when the displacement actuator is equipped with a capacitive measurement organ to determine the contact area between the displacement actuator and the tissue during engagement of the tissue by the displacement actuator.

Preferably the analyzer is arranged to derive an estimated stiffness value of the tissue from the estimated or measured contact area between the displacement actuator and the tissue during engagement of the tissue by the displacement actuator, or from a longitudinal comparison of the estimated or measured contact area developing over time and to derive from this comparison an indication of the presence of malignant cells.

Best results may be achieved when the estimation or measurement of the contact area between the displacement actuator and the tissue is monitored during a complete cycle of engaging and disengaging of the tissue by the displacement actuator.

In more advanced embodiments it is preferable that the analyzer is arranged to derive an estimated stiffness value of the tissue from a measured or calculated force applied to the tissue by the displacement actuator and the estimated or measured contact area between the displacement actuator and the tissue during engagement of the tissue by the displacement actuator.

In even further advanced embodiments it is preferable that the analyzer is arranged to derive an estimated stiffness value of the tissue from a longitudinal comparison of a measured or calculated force applied to the tissue by the displacement actuator over time, and the estimated or measured contact area developing over time between the displacement actuator and the tissue during engagement of the tissue by the displacement actuator.

There are embodiments in which the data processing device is equipped to determine from the data an estimated volume of the tissue being investigated. It may then or in other embodiments be preferable that the data processing device is equipped to determine from the data an estimated volume of the tissue being investigated when the displacement actuator is inactive and/or when the displacement actuator applies a load on the tissue and/or during a complete cycle of engaging and disengaging of the tissue by the displacement actuator.

Suitably the analyzer is arranged to derive an estimated stiffness value of the tissue from a measured or calculated force applied to the tissue and at least one of the estimated volume of the tissue, the contact area between the displacement actuator and the tissue, and a projected area obtained from a projection of the volume of the tissue, one thing and another derived from the data when the displacement actuator is inactive and/or when the displacement actuator applies a constant load to the tissue, and/or from the data when the displacement actuator applies an increasing load on the tissue. The term ‘projected area’ as used herein refers to a virtual contour projected in a line of sight from the detector to a supporting surface against which the displacement actuator presses the tissue under investigation.

It is further preferred that the system is equipped with a position sensor for measuring a position of a tissue engaging surface of the displacement actuator and/or a force sensor for measuring a force that the displacement actuator provides to the tissue, which position sensor and/or force sensor are connected to the data processing device for use by the data processing device in combination with the estimated or measured contact area between the displacement actuator and the tissue during engagement of the tissue by the displacement actuator so as to arrange that the load applied by the displacement actuator to the tissue is normalized to a pressure which the displacement actuator applies to the tissue.

Already mentioned above is a feasible option that the system is designed for processing data derived from a humans breast. It may then in particular be preferred that the system is designed for processing data derived from a left breast and a right breast from a same patient, and to arrange that the analyzer derives from the collected data certain differences between the left breast and the right breast which may support an indication of malignant cells in one breast or both breasts.

The invention will hereinafter be further elucidated with reference to the drawing of an exemplary embodiment of a system and method according to the invention that is not limiting as to the appended claims.

### BRIEF DESCRIPTION OF THE FIGURES

In the drawings:

FIG. 1 shows a basic layout of a system according to the present disclosure;

FIG. 2 shows a top view at the system of FIG. 1 during investigation; and

FIG. 3 shows a top view at the system of FIG. 1 during investigation of slightly deformed tissue caused by the presence of malignant cells.

Whenever in the figures the same reference numerals are applied, these numerals refer to the same parts.

### DETAILED DESCRIPTION

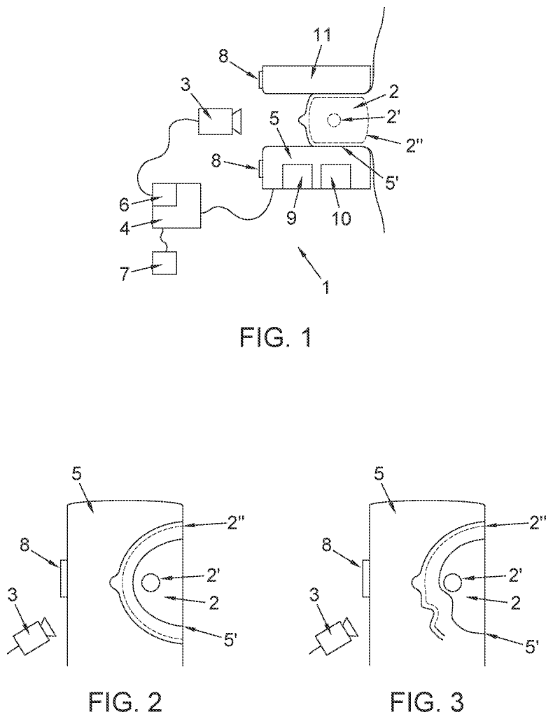

FIG. 1 shows a basic layout of a system 1 of the invention for screening tissue 2 on the presence of malignant cells in said tissue 2. In the shown case the tissue is of a breast, although this is not essential. Instead of a breast also other tissue may be screened with the system and method of the invention on the occurrence of malignant cells.

The system 1 comprises a wave detector 3 and a data processing device 4 connected or connectable to the wave detector 3 for processing data received from the wave detector 3.

The system 1 further comprises an actuator 5 to mechanically excite the tissue 2 which is suspected to comprise malignant cells, and the processing device 4 comprises an analyzer 6 connected to the wave detector 3 for analyzing the data received from the wave detector 3 in response to the actuator 5 mechanically exciting the tissue 2. The analyzer 6 is arranged to identify and select the tissue 2′ which has in comparison with tissue 2″ that is not suspected to comprise malignant cells, an elevated probability to comprise malignant cells.

The choice of wave detector 3 that is used is dependent on the type of actuator 5 that is applied to mechanically excite the tissue 2. Mechanically exciting the tissue 2 may be done for instance with a sound emitter, an ultrasound emitter, a pulsed laser source (which is known to inflict ultrasound waves in tissue subjected to the pulsed laser source), or—as is shown in FIG. 1—with a mechanical displacement actuator 5. FIG. 1 also shows a supporting surface 11 against which the displacement actuator 5 presses the tissue 2 under investigation.

Corresponding to the type of actuator used, the wave detector 3 may be selected as one of an (ultra-) sound wave detector, a visual light spectrum 2D or 3D static picture camera, a visual light spectrum 2D or 3D moving picture camera, or even an infrared detector, an ultraviolet detector, a LIDAR, a radar, or a microwave antenna. When as is shown in FIG. 1, a camera is applied this preferably is a CCD-camera 3.

When the actuator is a displacement actuator 5 it must be arranged for displacing a tissue engaging surface or contact area 5′ of the displacement actuator 5 along a predetermined path and/or applying a predetermined load on the tissue 2 to be investigated. The resulting deformation of the tissue 2 can then be observed with the wave detector 3. Based thereon the analyzer 6 of the data processing device 4 determines from the data received from the wave detector 3 an estimated stiffness value of the tissue 2 under investigation, wherein the analyzer 6 is arranged to use this stiffness value as an indication for the presence or nonpresence of malignant cells.

A nonlimiting example that relates to the envisaged detection of malignant cells with the wave detector 3 is the following. For comparative purposes FIG. 3 differs from FIG. 2 to show that tissue 2 under investigation may behave differently depending on the location and nature of the presence of malignant cells 2′ in the tissue 2. In FIG. 2 the pressure applied to the tissue 2 homogeneously spreads through the tissue 2 under investigation, whereas in FIG. 3 there is an inhomogeneous spreading of the pressure applied to the tissue 2. This difference may serve as a visual indication which is detectable by the wave detector 3 that further investigation may be warranted as to the (non-)presence of malignant cells.

In a refined embodiment of the system 1 of the invention the analyzer 6 determines from the data received from the wave detector 3 local differences of estimated stiffness values of the tissue 2 under investigation, wherein the analyzer 6 is arranged to use these local differences of stiffness values as an indication for the presence or nonpresence of malignant cells. The local differences in stiffness value correspond to healthy tissue and tissue comprising malignant cells. The deformation of the tissue as shown in FIG. 3 is an indication of such local differences in stiffness value.

FIG. 1 further shows that the data processing device 4 comprises or connects to a data storage device 7 for storage of data received from the wave detector 3. This enables that the analyzer 6 can be equipped to operate on data received from the wave detector 3 and/or from the data storage device 7, wherein the data has different timestamps so as to enable a longitudinal comparison of data over time and to derive from this comparison an indication of the probable presence of malignant cells.

FIG. 1 depicts the situation that the tissue 2 under investigation is mechanically excited by a load applied by the displacement actuator 5 on the tissue 2. It is preferable however that the wave detector 3 is enabled to collect and provide data to the data processing device 4 from two different situations, notably one situation in which said data relates at least to the tissue 2 being disengaged and released from a load by the displacement actuator 5, and another situation in which the tissue 2 is being engaged and loaded by the displacement actuator 5, the latter preferably according to a predetermined trajectory until reaching a predetermined full load being applied to the tissue 2.

FIG. 1 further schematically shows that the displacement actuator 5 may be equipped with at least one marker 8 which enables position detection of the tissue engaging surface or contact area 5′ of the displacement actuator 5. The tissue engaging surface or contact area 5′ is also shown in the top view provided by FIG. 2. For the purpose of position detection of the tissue engaging surface or contact area 5′ it is preferred that the wave detector 3 is arranged to detect the position of the marker 8. Such a detectable marker 8 is preferably also provided on the supporting surface 11 against which the displacement actuator 5 presses the tissue 2 under investigation. This is shown in FIG. 1.

Most preferably the data collected and provided by the wave detector 3 to the data processing device 4 relates to a complete load-path-trajectory wherein the load provided by the displacement actuator 5 on the tissue 2 develops from no-load to the predetermined full load.

In certain embodiments it is useful that the displacement actuator 5 is equipped with a capacitive measurement organ (not shown) to determine the contact area 5′ (see FIG. 2) between the displacement actuator 5 and the tissue 2 during engagement of the tissue 2 by the displacement actuator 5. If such a capacitive measurement organ is not applied, the data processing device 4 can be equipped to estimate from the data the contact area between the displacement actuator 5 and the tissue 2 as it develops during engagement of the tissue 2 by the displacement actuator 5. In particular it may then be desirable that the analyzer 6 is arranged to derive an estimated stiffness value of the tissue 2 from the estimated or measured contact area 5′ between the displacement actuator 5 and the tissue 2 during engagement of the tissue 2 by the displacement actuator 5, or from a longitudinal comparison of the estimated or measured contact area 5′ developing over time and to derive from this comparison an indication of the presence of malignant cells. The reliability of the detection of malignant cells may be further improved by arranging that the estimation or measurement of the contact area 5′ between the displacement actuator 5 and the tissue 2 is monitored during a complete cycle of engaging and disengaging of the tissue 2 by the displacement actuator 5.

In another embodiment the analyzer 6 is arranged to derive an estimated stiffness value of the tissue 2 from a measured or calculated force applied to the tissue 2 by the displacement actuator 5 and the estimated or measured contact area 5′ between the displacement actuator 5 and the tissue 2 during engagement of the tissue 2 by the displacement actuator 5. More preferred is that the analyzer 6 is arranged to derive an estimated stiffness value of the tissue 2 from a longitudinal comparison of a measured or calculated force applied to the tissue 2 by the displacement actuator 5 over time, and the estimated or measured contact area 5′ developing over time between the displacement actuator 5 and the tissue 2 during engagement of the tissue 2 by the displacement actuator 5. In combination the force applied by the displacement actuator 5 and the estimated or measured contact area 5′, enables the calculation of an estimated or measured pressure applied to the tissue 2 which is a factor which can be taken into account in the estimation whether or not malignant cells are present in the tissue 2.

In still another embodiment the data processing device is equipped to determine from the data an estimated volume of the tissue being investigated. In this embodiment it is preferred that the data processing device 4 is equipped to determine from the data an estimated volume of the tissue 2 being investigated when the displacement actuator 5 is inactive and/or when the displacement actuator 5 applies a load on the tissue 2 and/or during a complete cycle of engaging and disengaging of the tissue 2 by the displacement actuator 5.

It is also possible to combine data and to arrange the analyzer 6 to derive an estimated stiffness value of the tissue 2 from a measured or calculated force applied to the tissue 2 and at least one of the estimated volume of the tissue 2, the contact area 5′ between the displacement actuator 5 and the tissue 2, and a projected area obtained from a projection of the volume of the tissue, one thing and another derived from the data when the displacement actuator 5 is inactive and/or when the displacement actuator 5 applies a constant load to the tissue 2, and/or from the data when the displacement actuator 5 applies an increasing load on the tissue 2. The term ‘projected area’ as used herein refers to a virtual contour projected in a line of sight from the detector to a supporting surface 11 against which the displacement actuator 5 presses the tissue 2 under investigation.

To improve accuracy the system 1 may be equipped with a position sensor 9 for measuring a position of a tissue engaging surface 5′ of the displacement actuator 5 and/or a force sensor 10 for measuring a force that the displacement actuator 5 provides to the tissue 2, which position sensor 9 and/or force sensor 10 are connected (not shown) to the data processing device 4 for use by the data processing device 4 in combination with the estimated or measured contact area 5′ between the displacement actuator 5 and the tissue 2 during engagement of the tissue 2 by the displacement actuator 5 so as to arrange that the load applied by the displacement actuator 5 to the tissue 2 is normalized to a pressure which the displacement actuator 5 applies to the tissue 2.

Although this is not essential to the invention, FIG. 1 shows that the system 1 processes data derived from a humans breast. Under circumstances it may be preferable that the system 1 is designed for processing data derived from a left breast and a right breast from a same patient, and to arrange that the analyzer 6 derives from differences between the left breast and the right breast an indication of malignant cells in one breast or in both breasts.

Embodiments of the present invention can include every combination of features that are disclosed herein independently from each other. Although the invention has been discussed in the foregoing with reference to an exemplary embodiment of the system and method of the invention, the invention is not restricted to this particular embodiment which can be varied in many ways without departing from the invention. The discussed exemplary embodiment shall therefore not be used to construe the appended claims strictly in accordance therewith. On the contrary the embodiment is merely intended to explain the wording of the appended claims without intent to limit the claims to this exemplary embodiment. The scope of protection of the invention shall therefore be construed in accordance with the appended claims only, wherein a possible ambiguity in the wording of the claims shall be resolved using this exemplary embodiment.

Variations and modifications of the present invention will be obvious to those skilled in the art and it is intended to cover in the appended claims all such modifications and equivalents. The entire disclosures of all references, applications, patents, and publications cited above are hereby incorporated by reference. Unless specifically stated as being “essential” above, none of the various components or the interrelationship thereof are essential to the operation of the invention. Rather, desirable results can be achieved by substituting various components and/or reconfiguration of their relationships with one another.

Optionally, embodiments of the present invention can include a general or specific purpose computer or distributed system programmed with computer software implementing steps described above, which computer software may be in any appropriate computer language, including but not limited to C++, FORTRAN, ALGOL, BASIC, Java, Python, Linux, assembly language, microcode, distributed programming languages, etc. The system may also include a plurality of such computers/distributed systems (e.g., connected over the Internet and/or one or more intranets) in a variety of hardware implementations. For example, data processing can be performed by an appropriately programmed microprocessor, computing cloud, Application Specific Integrated Circuit (ASIC), Field Programmable Gate Array (FPGA), or the like, in conjunction with appropriate memory, network, and bus elements. One or more processors and/or microcontrollers can operate via instructions of the computer code and the software is preferably stored on one or more tangible non-transitive memory-storage devices.

What is claimed is:

1. A system for screening tissue on the presence of malignant cells in said tissue, which system comprises:
a wave detector; and
a data processing device connected or connectable to the wave detector for processing data received from the wave detector;
wherein, the system comprises an actuator to mechanically excite the tissue which is suspected to comprise malignant cells, and the data processing device comprises an analyzer connected to the wave detector for analyzing the data received from the wave detector in response to the actuator mechanically exciting the tissue, which analyzer is arranged to identify the tissue with an elevated probability to comprise malignant cells in comparison with tissue that is not suspected to comprise malignant cells.

2. The system of claim 1, wherein the analyzer determines from the data received from the wave detector an estimated stiffness value of the tissue under investigation, wherein the analyze is arranged to use this stiffness value as an indication for the presence or nonpresence of malignant cells.

3. The system of claim 2, wherein the analyzer determines from the data received from the wave detector local differences of estimated stiffness values of the tissue under investigation, wherein the analyzer is arranged to use these local differences of stiffness values as an indication for the presence or nonpresence of malignant cells.

4. The system of claim 1, wherein the data processing device comprises or connects to a data storage device for storage of data received from the wave detector.

5. The system of claim 1, wherein the analyzer is equipped to operate on data received from the wave detector and/or from the data storage device, wherein the data has different timestamps so as to enable a longitudinal comparison of data over time and to derive from this comparison an indication of the probable presence of malignant cells.

6. The system of claim 1, wherein the actuator is one of a sound emitter, an ultrasound emitter, a pulsed laser source, a displacement actuator.

7. The system of claim 6, wherein the displacement actuator is equipped with at least one marker which enables position detection of the tissue engaging surface or contact area of the displacement actuator, and that preferably a further detectable marker is provided on the supporting surface against which the displacement actuator presses the tissue under investigation.

8. The system of claim 1, wherein the actuator is a displacement actuator for displacing a tissue engaging surface along a predetermined path and/or applying a predetermined load on the tissue to be investigated.

9. The system of claim 8, wherein the wave detector is arranged to collect and provide data to the data processing device, said data relating at least to the tissue being disengaged and released from a load by the displacement actuator, and to the tissue being engaged and loaded by the displacement actuator to a predetermined full load.

10. The system of claim 8, wherein the data processing device is equipped to estimate from the data a contact area between the displacement actuator and the tissue during engagement of the tissue by the displacement actuator.

11. The system of claim 8, wherein the displacement actuator is equipped with a capacitive measurement organ to determine a contact area between the displacement actuator and the tissue during engagement of the tissue by the displacement actuator.

12. The system of claim 8, wherein the analyzer is arranged to derive an estimated stiffness value of the tissue from a measured or calculated force applied to the tissue by the displacement actuator and the estimated or measured contact area between the displacement actuator and the tissue during engagement of the tissue by the displacement actuator.

13. The system of claim 8, wherein the analyzer is arranged to derive an estimated stiffness value of the tissue from a longitudinal comparison of a measured or calculated force applied to the tissue by the displacement actuator over time, and the estimated or measured contact area developing over time between the displacement actuator and the tissue during engagement of the tissue by the displacement actuator.

14. The system claim 8, wherein the system is equipped with a position sensor for measuring a position of a tissue engaging surface or contact area of the displacement actuator and/or a force sensor for measuring a force that the displacement actuator provides to the tissue, which position sensor and/or force sensor are connected to the data processing device for use by the data processing device in combination with the estimated or measured contact area between the displacement actuator and the tissue during engagement of the tissue by the displacement actuator so as to arrange that the load applied by the displacement actuator to the tissue is normalized to a pressure which the displacement actuator applies to the tissue.

15. The system of claim 10, wherein the analyzer is arranged to derive an estimated stiffness value of the tissue from the estimated or measured contact area between the displacement actuator and the tissue during engagement of the tissue by the displacement actuator, or from a longitudinal comparison of the estimated or measured contact area developing over time and to derive from this comparison an indication of the presence of malignant cells.

16. The system of claim 10, wherein the estimation or measurement of the contact area between the displacement actuator and the tissue is monitored during a complete cycle of engaging and disengaging of the tissue by the displacement actuator.

17. The system of claim 10, wherein the analyzer is arranged to derive an estimated stiffness value of the tissue from a measured or calculated force applied to the tissue and at least one of the estimated volume of the tissue, the contact area between the displacement actuator and the tissue, and a projected area obtained from a projection of the volume of the tissue, one thing and another derived from the data when the displacement actuator is inactive and/or when the displacement actuator applies a constant load to the tissue, and/or from the data when the displacement actuator applies an increasing load on the tissue.

18. The system of claim 1, wherein the wave detector is at least one of an (ultra-) sound wave detector, a visual light spectrum 2D or 3D static picture camera, a visual light spectrum 2D or 3D moving picture camera, an infrared detector, an ultraviolet detector, a LIDAR, a radar, a microwave antenna.

19. The system of claim 18, wherein the camera is a CCD-camera.

20. The system of claim 18, wherein the wave detector is arranged to detect the position of the marker.
